(19) United States
(12) Patent Application Publication
(10) Pub. No.: US 2025/0367439 A1
(43) Pub. Date: Dec. 4, 2025

(54) NEURAL IMPLANT SYSTEM AND METHOD

(71) Applicant: Capri Medical Limited, Dublin (IE)

(72) Inventors: Fergal Ward, Dublin (IE); John Mullins, Dublin (IE); Ross Lewis, Dublin (IE)

(73) Assignee: Capri Medical Limited, Dublin (IE)

(21) Appl. No.: 18/874,846

(22) PCT Filed: Jun. 15, 2023
(86) PCT No.: PCT/EP2023/066173

§ 371 Date: Dec. 13, 2024

Related U.S. Application Data
(60) Provisional application No. 63/352,353, filed on Jun. 15, 2022.

(51) Int. Cl. A61N 1/05; A61B 17/34

(52) U.S. Cl. CPC A61N 1/0558; A61B 17/3468

(57) ABSTRACT

A neural implant system for percutaneous delivery of a neural implant (1) in a patient's tissue, the neural implant system includes a neural implant (1) having a housing portion (2) and an elongate electrode lead (3), and a delivery device (10) having a handle (11), a first needle (12) fixed to the handle and having a lumen adapted to receive the housing portion of the neural implant, a second needle (13) having a higher gauge than the first needle, the second needle comprising a retaining portion partially surrounding the elongate electrode lead and an open side extending at least partially along the length of the second needle, wherein the second needle is retractably mounted to the housing and retractable to deploy the electrode lead in the patient's tissue, wherein the electrode lead includes a resiliently biased anti-migration member (32) aligned with the retaining portion of the second needle so as to be constrained before deployment, and wherein the resiliently biased anti-migration member is configured to move into a deployed position after retraction of the second needle.

This invention relates to a neural implant system for percutaneous delivery of a neural implant into a patient's tissue, a method of percutaneously delivering a neural implant, and a medical implant.

### BACKGROUND

It is known to provide an implantable neurostimulator comprising a housing and an electrode. A power antenna, microcontroller, and communication antenna are disposed in the housing for receiving power from an external source and receiving/transmitting sensor information relating to the electrode. A delivery system can be used to position the neurostimulator in a patient, in particular proximate to a nerve, by cutting an opening in the patient and passing the delivery system into the opening to position the implantable neurostimulator.

### BRIEF SUMMARY OF THE DISCLOSURE

In accordance with the present disclosure there is provided a neural implant system for percutaneous delivery of a neural implant in a patient's tissue, the neural implant system comprising:
a neural implant having a housing portion and an elongate electrode lead, and
a delivery device comprising:
   a handle,
   a first needle fixed to the handle and having a lumen adapted to receive the housing portion of the neural implant,
   a second needle having a higher gauge than the first needle, the second needle comprising a retaining portion partially surrounding the elongate electrode lead and an open side extending at least partially along the length of the second needle, wherein the second needle is retractably mounted to the housing and retractable to deploy the electrode lead in the patient's tissue,
wherein the electrode lead comprises a resiliently biased anti-migration member aligned with the retaining portion of the second needle so as to be constrained before deployment, and wherein the resiliently biased anti-migration member is configured to move into a deployed position after retraction of the second needle.

In examples, the resiliently biased anti-migration member is a resiliently deformable anti-migration member.

In examples, the retaining portion of the second needle may comprise a wall having a recess arranged to receive the resiliently biased anti-migration member. In examples, the recess may be an opening extending through the wall of the retaining portion.

In examples, the electrode lead may comprise a plurality of resiliently biased anti-migration members.

In examples, the delivery device may comprise a pusher operable to push the housing portion out of the first needle.

In examples, the neural implant may comprise one or more resiliently biased anti-migration members disposed on the housing portion. In examples, the first needle may comprises a wall having a recess arranged to receive the resiliently biased anti-migration member.

In examples, the electrode lead may comprise an electrode, and the resiliently biased anti-migration member may be disposed on the electrode lead between the electrode and the housing portion.

In examples, the or each resiliently biased anti-migration member may comprise a fin. The fin may be foldable against the neural implant. The fin may be shaped to be angled towards the skin of the user when implanted.

In examples, the resiliently biased anti-migration member may comprise a tine. The resiliently biased anti-migration member may comprise a plurality of tines. In examples, a first tine of the plurality of tines may be directed in an opposite direction to a second tine of the plurality of tines.

In examples, the resiliently biased anti-migration member may comprise a hook. In examples, the hook may comprise a shape-memory material, for example a shape-memory metal alloy, such as Nitinol, or a shape-memory polymer. In examples, the hook may be directed towards the housing portion.

In examples, the resiliently biased anti-migration member may comprise a stent. In examples, the stent may comprise a collapsible frame that is resiliently biased to an expanded position.

In examples, the resiliently biased anti-migration member may comprise a coil, or a plurality of coils.

In examples, the resiliently biased anti-migration member may be disposed at or near a distal tip of the elongate electrode lead.

In examples, the resiliently biased anti-migration member may comprise a shape-memory member embedded within the electrode lead. The shape-memory member may be resiliently biased to a non-linear form. The second needle may retain the elongate electrode lead in a substantially linear form before retraction of the second needle.

In examples, the second needle comprises an opening extending longitudinally along one side of the second needle. In such examples, the resiliently biased anti-migration member may comprise a tine disposed on the electrode lead, and a width of the tine may be greater than a width of the opening in the second needle. In other such examples, the resiliently biased anti-migration member may comprise a tine disposed on the electrode lead, and the tine may be arranged so as not to align with the opening of the second needle.

In examples, the resiliently biased tine has a first end attached to the electrode lead and a second end that is a free end. A width of the resiliently biased tine at the second end may be greater than a width of the resiliently biased tine at the first end. In examples, the resiliently biased tine is curved.

In various examples, the resiliently biased anti-migration member comprises a sleeve portion surrounding a part of the electrode lead to secure the resiliently biased anti-migration member to the electrode lead. The sleeve portion may be attached to the electrode lead, for example by adhesive, welding, crimping, or friction fit. In some examples, the sleeve portion may be overmoulded on the electrode lead.

In examples, the resiliently biased anti-migration member may comprise one or more openings formed therein for tissue ingrowth after implantation.

In examples, the second needle and the electrode lead comprise anti-rotation features. The anti-rotation features co-operate to prevent rotation of the electrode lead within the second needle. The anti-rotation features may extend partially or fully along the second needle, and/or partially or fully along the electrode lead. Preferably, the anti-rotation member on the electrode lead is aligned with the resiliently biased anti-migration member. In examples, the anti-rotation features comprise opposing flat surfaces that abut to prevent rotation. In examples, the anti-rotation feature comprise a protrusion and slot that receives the protrusion to prevent rotation.

In examples, the electrode lead further comprises one or more ridged portions.

According to a further aspect of the present invention there is also provided a method of percutaneously delivering a neural implant into tissue of a patient, the method comprising:
providing the neural implant system described above,
percutaneously positioning the second needle in the patient's tissue to a desired anatomical position,
retracting the second needle to release the electrode lead into the tissue, and
deploying the anti-migration member.

According to a further aspect of the present invention there is also provided a medical implant for percutaneous implantation into a patient's tissue by a delivery device, the medical implant comprising a resiliently biased anti-migration member adapted to move from a retracted position when the medical implant is received in the delivery device, to a deployed position when the medical implant is released from the delivery device.

In examples, the medical implant may further comprise a housing portion and an elongate electrode lead extending from the housing portion. In examples, the resiliently biased anti-migration member is disposed on the electrode lead and/or on the housing portion.

In various examples, the resiliently biased anti-migration member may comprise one or more of:
a fin protruding from the medical implant,
a tine protruding from the medical implant,
a hook protruding from the medical implant,
a stent protruding from the medical implant,
a coil protruding from the medical implant, and/or
a shape-memory member embedded within the electrode lead.

According to a further aspect of the present invention there is also provided a delivery device for percutaneously implanting a neural implant in a patient's tissue, wherein the neural implant has an housing portion and an elongate electrode lead extending from the housing portion, and wherein the delivery device comprises:
a handle,
a first needle fixed to the handle and having a lumen adapted to receive the housing portion of the neural implant,
a second needle having a higher gauge than the first needle, the second needle comprising a retaining portion partially surrounding the elongate electrode lead and an open side extending at least partially along the length of the second needle, wherein the second needle is retractably mounted to the housing and retractable to deploy the electrode lead in the patient's tissue, and
wherein the retaining portion comprises a recess to accommodate a resiliently biased anti-migration member of the neural implant within the second needle.

### BRIEF DESCRIPTION OF THE DRAWINGS

Embodiments of the invention are further described hereinafter with reference to the accompanying drawings, in which:

FIG. 1 shows a medical implant;

FIG. 2 show the medical implant in position in a patient, particularly in a subcutaneous position;

FIGS. 3A to 3C show alternative medical implants;

FIG. 4 shows a schematic cross-section of the delivery device, including a handle, in a configuration before implanting the medical implant;

FIGS. 5A to 5C illustrate operation of the delivery device to implant the medical implant;

FIG. 6 shows a perspective view of an end of the first needle and the second needle, and a medical implant retained by the first and second needles;

FIG. 7A shows the second needle;

FIG. 7B shows the first needle;

FIG. 8 shows an example implant delivery device in which the second needle is initially in a retracted position;

FIG. 9 shows an example implant delivery device in which the tab is moved towards the handle to retract the second needle;

FIGS. 10 to 31 show different example anti-migration members of the medical implant;

FIGS. 32 and 33 illustrate examples of a medical implant and needle with an anti-rotation feature; and

FIGS. 34 and 35 illustrate example electrode leads of medical implants that include ridged portions.

### DETAILED DESCRIPTION

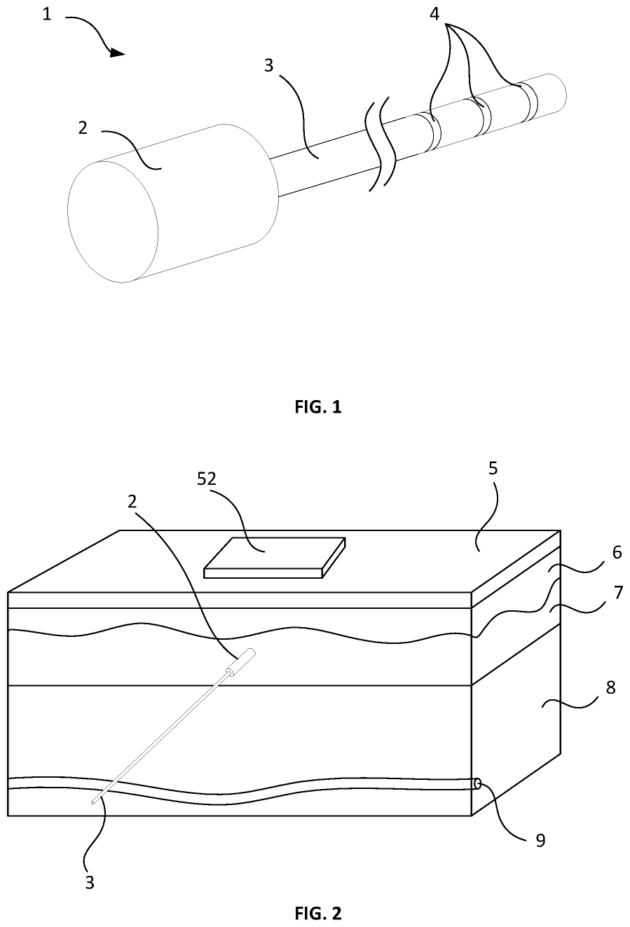

FIG. 1 schematically illustrates a medical implant 1. In examples, the medical implant 1 may a neural implant, for example a neurostimulator implant or a diagnostic implant. The medical implant 1 comprises a housing portion 2 and an elongate electrode lead 3. The housing portion 2 may house electronics components of the medical implant 1, including for example a printed circuit board, a wireless communications receiver/transmitter, a wireless power receiver, and/or sensor electronics, as described further hereinafter. In examples, the housing portion is hermetically sealed. The housing portion 2 may comprise a cylindrical casing with sealed ends, or may comprise a wrapping or other envelopment of the components within the housing portion 2.

In examples, the electrode lead 3 extends from the housing portion 2 and is flexible. The electrode lead 3 includes at least one electrode 4, in some examples multiple electrodes 4 spaced along the length of the electrode lead 3. The electrodes 4 are connected to the electronics within the housing portion 2.

In examples, the housing portion 2 may have a diameter of between about 0.5 millimetres and about 5 millimetres, for example between about 1 millimetre and about 3 millimetres. The housing portion 2 may have a length of up to about 10 millimetres, for example up to about 5 millimetres. In examples, the electrode lead 3 may have a diameter of between about 0.3 millimetres to about 1.5 millimetres, for example between about 0.5 millimetres and 1.3 millimetres. The electrode lead 3 may have a length of up to about 100 millimetres, for example up to about 50 millimetres, for example about 50 millimetres. However, it will be appreciated that the dimensions of the housing portion 2 would correspond to the size of the electronics housed within the housing portion 2, and the length of the electrode lead 3 would correspond to the anatomy surrounding the targeted nerve, so a shorter or longer electrode lead 3 may be appropriate depending on the depth of the nerve within the muscle tissue

As described further hereinafter, the medical implant 1 is implantable in a patient and operable to sense and/or stimulate a nerve. In some examples, the medical implant 1 is implantable to sense and/or stimulate the greater occipital nerve, although the same or similar implant may be implantable to sense and/or stimulate other nerves, particularly other peripheral nerves of the peripheral nervous system. In examples, the medical implant 1 may be implantable to sense and/or stimulate the tibial nerve, the sacral nerve (e.g., to treat urinary incontinence) or the vagus nerve (e.g., to regulate pancreatic secretion).

FIG. 2 shows the medical implant 1 once implanted in a patient. The medical implant 1 is positioned below the surface of the skin, in particular below the epidermis 5. The housing portion 2 may be positioned in the dermis 6 or in the subcutaneous tissue 7. Positioning the housing portion 2 in the subcutaneous tissue 7 may be beneficial to cause less damage and/or irritation to the patient.

As illustrated, the electrode lead 3 extends from the housing portion 2, through the underlying tissue, in particular muscle 8, to a position proximal to the target nerve 9. The electrode lead 3 is positioned such that the electrodes (4, see FIG. 1) are in contact with or proximal to the nerve 9, and so the electrodes 4 can be used to sense and/or stimulate the nerve 9.

The medical implant 1 may also include one or more anti-migration members. The anti-migration members may be provided on the housing portion 2 and/or on the electrode lead 3 and function to hold the medical implant 1 in position in the patient's tissue.

In examples, the medical implant 2 is battery-less, and does not have an integrated power source. An external device 52 can wirelessly power the medical implant 1. The external device 52 may additionally wirelessly communicate with the medical implant 1, in particular the electronics in the housing portion 2. The medical implant 1 may contain a wireless communications receiver/transmitter for communicating with the external device 52. The medical implant 1 may also have a processor or controller configured to operate the medical implant 1. The external device 52 may be positioned on the skin proximal to the medical implant 1. The external device 52 may be adhered to the skin proximal to the medical implant 1. The external device 52 may be a wearable device.

In examples, the medical implant 1 is a neural implant. The medical implant 1 may be implanted to target a particular nerve or nerve grouping, such as the greater occipital nerve.

In operation, the electrodes 4 of a neurostimulator implant are provided with an electrical signal, such as a current, to stimulate the nerve. In examples, the electrical signal may be a voltage-regulated stimulation. Such stimulation can provide relief for chronic pain, for example occipital neuralgia, intractable migraine, and/or other therapeutic benefits.

In other examples, the medical implant 1 may be a diagnostic implant, for example a neurodiagnostic implant, operable to detect one or more neural signals in a nerve. In such examples the electrodes 4 are operable to detect neural signals. The neural signals may be analysed for the purposes of detecting, monitoring and/or diagnosing a condition.

In other examples, the diagnostic implant may additionally or alternatively detect one or more patient vital signs, for example body temperature, heart rate, electromyography (EMG), electrocardiogram (ECG), respiration rate, blood pressure, and/or blood gas concentration (e.g., oxygen, carbon dioxide, carbon monoxide).

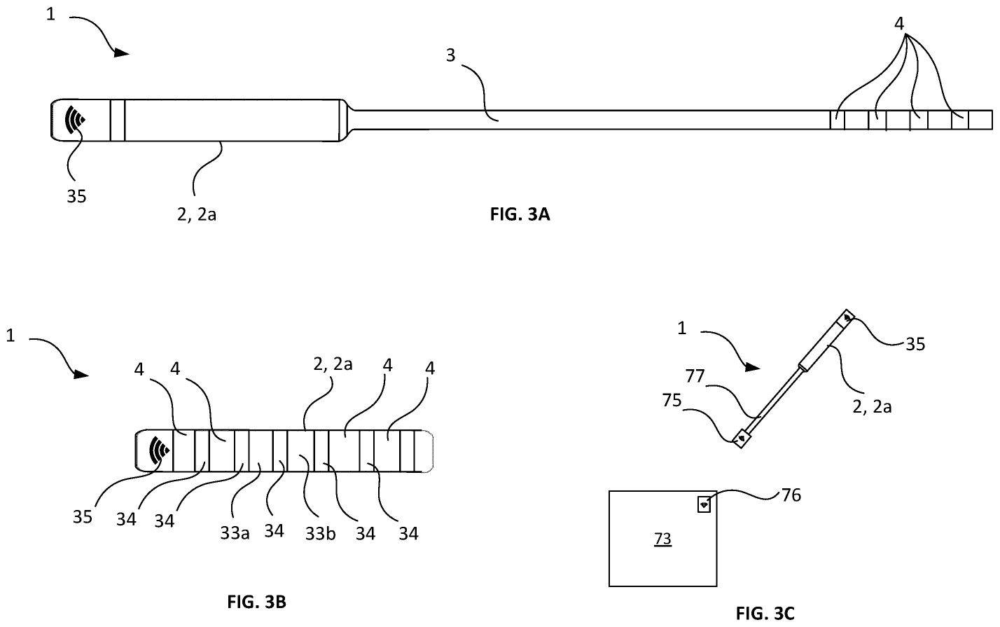

FIGS. 3A to 3C illustrate alternative examples of the medical implant 1.

In the example of FIG. 3A the medical implant 1 includes a housing portion 2 and an electrode lead 3 with electrodes 4 formed on the electrode lead 3. The housing portion 2 comprises a casing 2a. The casing 2a may be cylindrical and house electronic components within. The housing portion 2 comprises a wireless power receiver 35 for receiving wireless power from an external device (52, see FIG. 2) once implanted in the patient.

In the example of FIG. 3B the medical implant 1 does not comprise an electrode lead 3, and the electrodes 4 are formed on the housing portion 2. The example of FIG. 3B may be a neural implant, for example a neurostimulator implant or diagnostic implant implantable proximate to a nerve in the same manner as described above. In this example, the casing 2a of the housing portion 2 comprises the electrodes 4, separated by insulated areas 34. In some examples, as illustrated, the housing portion 2 also holds a wireless power receiver 35. The wireless power receiver 35 is disposed in the housing portion 2 and may be spaced from the electrodes 4.

In the example of FIG. 3C the medical implant 1 is for use in conjunction with a further medical implant 73. The further medical implant 73 may be a deep tissue implant, for example a pacemaker. In these examples, the medical implant 1 can act as a wireless power receiver or as a wireless power relay for the further medical implant 73. The medical implant comprises a wireless power receiver 35 and a wireless power transmitter 75 and is arranged to relay power to a wireless power receiver 76 of the further medical implant 73. The wireless power transmitter 75 of the medical implant 1 may be connected to the housing portion 2 by a wire 77, as illustrated, or it may be within the housing portion 2 and the wire 77 may be omitted. The medical implant 1 can be implanted at a lower depth in the tissue than the further medical implant 73, providing improved wireless power coupling.

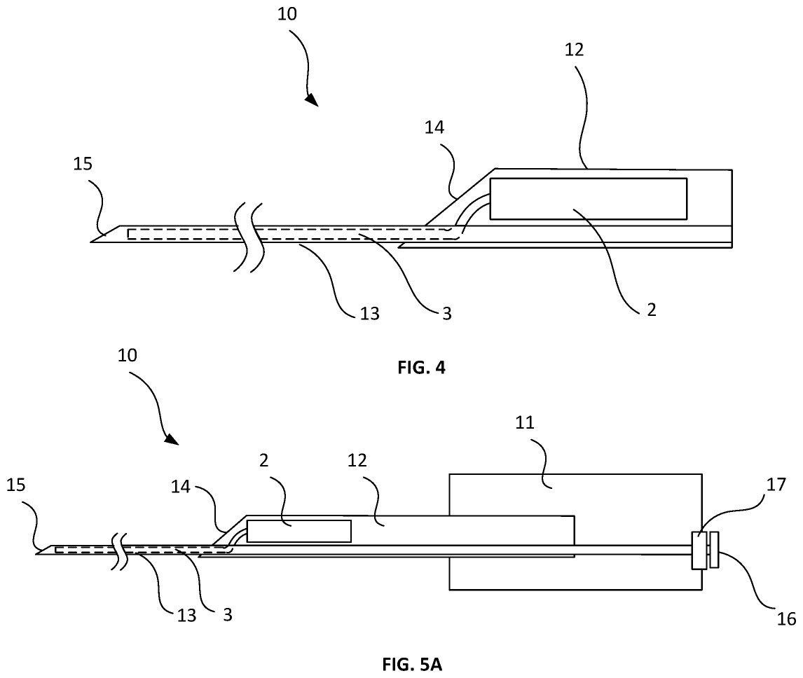

FIG. 4 illustrates an example delivery devices 10 for implanting a medical implant 1 in a patient's tissue. In particular, the delivery device 10 is used to percutaneously implant the medical implant 1 to the location shown in FIG. 2. The delivery device 10 is illustrated with reference to an example medical implant 1, specifically a neurostimulator implant, having a housing portion 2 and an electrode lead 3 as illustrated in FIG. 3A. However, it will be appreciated that the delivery devices 10 may be adapted for implantation of other medical implants 1 described with reference to FIGS. 3A to 3C.

In example of FIG. 4 the delivery device 10 has a delivery sheath comprising a first part and a second part, specifically a first needle 12 and a second needle 13. The first and second needles 12,13 are parallel and both extend in a longitudinal direction. The second needle 13 extends further than the first needle 12. In particular, the first needle 12 has a tip 14, such as a bevel tip, and the second needle 13 extends past the tip 14 of the first needle 12. The second needle 13 also has a tip 15, in particular a bevel tip. The second needle 13 has a higher gauge than the first needle 12 (i.e., the second needle 13 has a smaller diameter than the first needle 12).

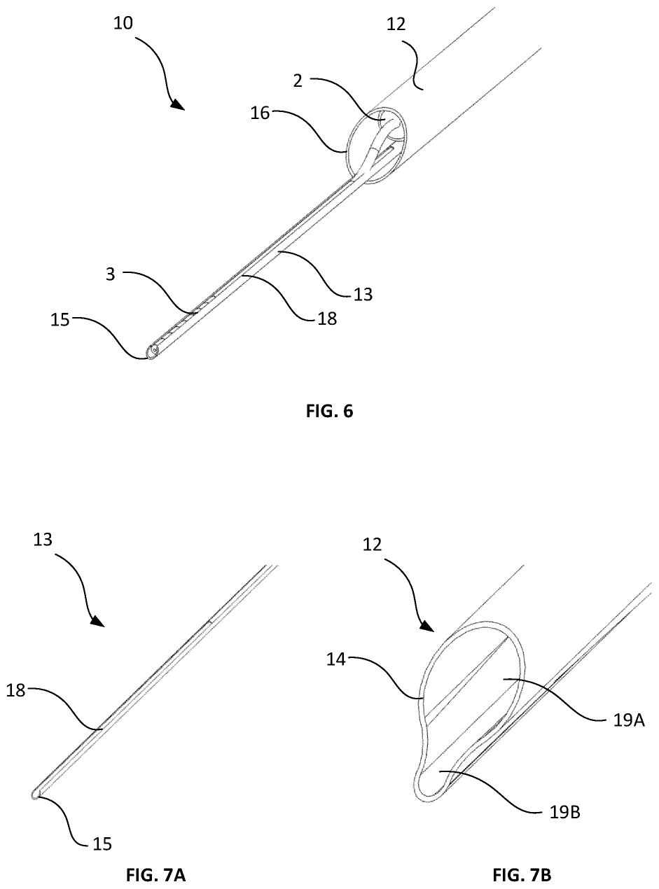

During use, the housing portion 2 of the medical implant 1 is received in the first needle 12, in particular in a lumen of the first needle 12. During use, the electrode lead 3 is received in the second needle 13, in particular in a lumen of the second needle 13. The electrode lead 3 extends along a substantial part of the second needle 13 towards the tip 15, as illustrated. A part of the electrode lead 3 adjacent to the housing portion 2 extends through an opening in the second needle 13, as described further with reference to FIG. 6. Accordingly, during use the medical implant 1 is housed within the first and second needles 12, 13 of the delivery device 10.

As illustrated, the first needle 12 and the second needle 13 are axially offset. In particular, a central axis of the first needle 12 is offset from a central axis of the second needle 13. In the illustrated example the second needle 13 extends into the first needle 12 (in particular into the lumen of the first needle 12), such that the second needle 13 is partly accommodated within the first needle 12 alongside the housing portion 2.

Referring to FIG. 6, the second needle 13 includes an opening 18, or slot, to permit a part of the electrode lead 3 to extend out of the second needle 13 and connect to the housing portion 2. The opening 18 may extend to the tip 15 of the second needle 13. The opening 18 may extend along the majority of the second needle 13, or all of the second needle 13.

Referring again to FIG. 4, during use the first needle 12 percutaneously penetrates the patient's tissue to position the housing portion 2 at a first depth, and the second needle 13 percutaneously penetrates the patient's tissue to position the electrode lead 3 at a second depth. Once the electrode lead 3 is correctly positioned the delivery device 10 then releases and deploys the medical implant 1 to leave the medical implant 1 in the position illustrated in FIG. 2. The tips 14, 15 of the first and second needles 12, 13, respectively, are sharp tips adapted to pierce the patient's skin and penetrate the tissue to percutaneously position the first and second needles 12, 13 at the appropriate depth. The tips 14, 15 may be bevel tips, as would be known to the skilled person. In some examples, during use the first and second needles 12,13 are used to pierce the patient's skin and underlying tissue, and in some examples an incision is first made and the first and second needles 12, 13 are inserted through the incision into the patient's tissue.

In examples, the first needle 12 may have a gauge of between 6 gauge and 15 gauge, for example 10 gauge. In examples, the second needle 13 may have gauge of between 15 gauge and 25 gauge, for example 20 gauge.

In various examples, the second needle 13 is retractable relative to the first needle 12, to deploy the electrode lead 3. The opening 18 (see FIG. 6) along the second needle 13 permits deployment of the electrode lead 3 as the second needle 13 is retracted.

In examples, the housing portion 2 is releasably attached to the first needle 12 (or another part of the delivery device 10) and is released prior to deployment.

In examples, the housing portion 2 may be deployed from the first needle 12 simply by pulling the implant delivery device away from the patient and relying on friction between the electrode lead 3 and the patient's tissue to hold the medical implant in place and pull the housing portion 2 from the first needle 12. In other examples, the delivery device 10 may include a deployment member, such as a pusher, adapted to push the housing portion 2 out of the first needle 12 to deploy the housing portion 2 at the appropriate anatomical site. In some examples, the implant delivery device may include a retaining member arranged to hold the housing portion 2 in the first needle 12 prior to deployment, and may be operable to release the housing portion 2 before the delivery device 10 is removed.

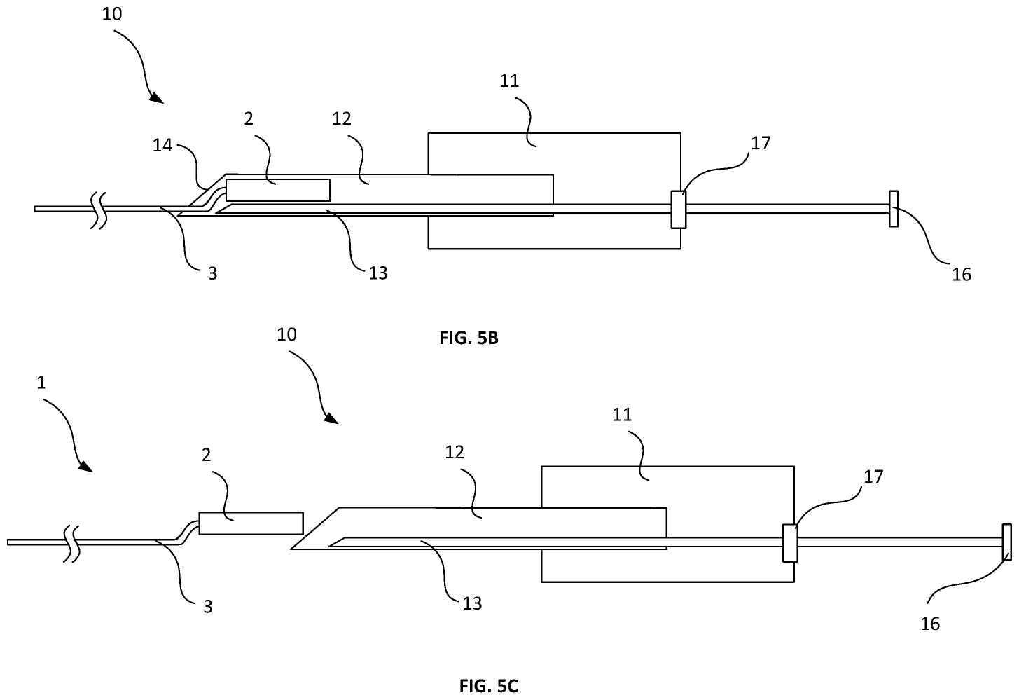

FIGS. 5A to 5C illustrate operation of the delivery device 10 of FIG. 4.

As illustrated, the example delivery device 10 further includes a handle 11. The handle 11 is adapted to be held by an operator. The first needle 12 is fixed to the handle 11.

The second needle 13 extends through the first needle 12 and through the handle 11. An actuation tab 16 is provided on an end of the second needle 13 opposite to the tip 15. In particular, the actuation tab 16 may be a gripping handle or similar for the operator to grip.

The second needle 13 is retractable relative to the first needle 12. In particular, the second needle 13 can slide through the first needle 12 and handle 11, from the position shown in FIG. 5A to the position shown in FIG. 5B. In this example, the second needle 13 is retractable by pulling the actuation tab 16 in a direction away from the patient. Retracting the second needle 13 in this way deploys the electrode lead 3. The opening 18 (see FIG. 6) along the second needle 13 provides for the part of the electrode lead 3 that connects to the housing portion 2. The opening 18 extends to the tip 15 of the second needle 13.

A locking device 17 is provided to lock the second needle 13 to the handle 11 and/or first needle 12. As shown, the locking device 17 may be provided at or near the actuation tab 16, and in examples locks the actuation tab 16 and/or the second needle 13 to the handle 11. The locking device 17 locks the second needle 13 in the extended position shown in FIG. 5B.

In the position shown in FIG. 5A the operator can percutaneously position the implant delivery device 10 in the patient's tissue. The position of the second needle 13 is locked relative to the handle 11, so the operator can push the implant delivery device 10 into the patient by the handle 11.

Referring to FIGS. 5A and 2, as the implant delivery device 10 is pushed into the patient the second needle 13 first penetrates the skin 5, and as the implant delivery device 10 is pushed further the first needle 12 then penetrates the skin 5. The first and second needles 12, 13 are thereby positioned at the appropriate depths in the patient, and the housing portion 2 and electrode lead 3 are also simultaneously positioned at the appropriate depths. Therefore, the implant delivery device 10 provides percutaneous delivery of the medical implant 1 into the patient's tissue.

In examples, the operator may use an ultrasound imaging device to monitor the positions of the second needle 13 (and the first needle 12) to guide the second needle 13 towards the target nerve 9.

Once the implant delivery device 10 is in position, with the tip 15 of the second needle 13 (and the electrode lead 3 within the second needle 13) being positioned proximate to the nerve, and the tip 14 of the first needle 12 (and the housing portion 2 within the first needle 12) being positioned in the subcutaneous tissue, the second needle 13 can be partially retracted to a position between those shown in FIGS. 5A and 5B. In particular, the second needle 13 can be partially retracted to expose the electrodes (4, see FIG. 3A). In this position the position of the electrodes (4, see FIG. 3A) can be tested. If needed, the second needle 13 can be re-extended to the position shown in FIG. 6A and repositioned.

Once the electrode lead 3 is appropriately positioned, the second needle 13 is retracted to the position shown in FIG. 5B to deploy the electrode lead 3.

The second needle 13 is retracted by unlocking the locking mechanism 17 and pulling on the actuation tab 16 relative to the handle 11 to slide the second needle 13 to the retracted position shown in FIG. 5B. The actuation tab 16 is pulled in a direction away from the patient. During retraction of the second needle 13 the handle 11 and first needle 12 remain stationary. As shown, once the second needle 13 is retracted the electrode lead 3 is deployed and exposed to the surrounding tissue (and nerve).

Next, as shown in FIG. 5C, the implant delivery device 10 is withdrawn from the patient and the housing portion 2 is deployed from the first needle 12. In this example the friction between the electrode lead 3 and the tissue (see muscle 8 in FIG. 2) is enough to hold the medical implant 1 in place as the implant delivery device 10 is withdrawn, thereby deploying the housing portion 2 out of the first needle 12 as the implant delivery device 10 is withdrawn from the patient.

In other examples the delivery device 10 may include a deployment member (e.g., a pusher in the first needle 12) configured to push the housing portion 2 out of the first needle 12. In some examples, the implant delivery device 10 may have a retaining member arranged to releasably attach the housing portion 2 to the first needle 12 and/or handle 11, and the retaining member can release the housing portion 2 after the second needle 13 is retracted and before the first needle 12 is removed from the patient.

FIGS. 6, 7A and 7B illustrate the first needle 12 and the second needle 13 of the delivery device 10 described above. As shown, the electrode lead 3 is received in the second needle 13, and the second needle 13 includes a slot 18 that allows the electrode lead 3 to connect to the housing portion 2 in the first needle 12. The slot 18 extends partially along the second needle 13 from the tip 15, and may extend the majority of the length of the second needle 13, or the entire length of the second needle 13. The second needle 13 may be in the form of a sheath. The first needle 12 receives the housing portion 2.

The first needle 12 includes a bevel tip 14. The second needle 13 includes a bevel tip 15. The bevel tips 14, 15 are sharp for piercing a patient's skin and penetrating the tissue during use.

As shown in FIG. 6, the second needle 13 extends through the lumen of the first needle 12. In particular, as shown in FIG. 7B the first needle 12 includes a primary portion 19A and a secondary portion 19B. The primary and secondary portions 19A, 19B are merged such that the first needle 12 has a single lumen. The primary and secondary portions 19A, 19B are shaped to define the two distinct portions 19A, 19B.

The primary portion 19A is shaped to receive the housing portion 2. In particular, the primary portion 19A is sized to receive the housing portion 2 and has a substantially circular cross-section that retains the housing portion 2 in axial alignment within the primary portion 19A.

The secondary portion 19B is shaped to receive the second needle 13. In particular, the secondary portion 19B is sized to receive the second needle 13 and has a substantially circular cross-section that retains the second needle 13 in axial alignment within the secondary portion 19B.

In examples, the secondary portion 19B and the primary portion 19A each have a substantially circular cross-section, and the cross-sections at least partially overlap. In such an example, the housing portion may be pushed over to one side of the primary portion 19A by the presence of the second needle 13 in the secondary portion 19B.

The slot 18 in the second needle 13 is directed towards the centre of the first needle 12, allowing the electrode lead 3 to connect with the housing portion 2 as shown in FIG. 6.

Accordingly, the first needle 12 is shaped to receive the housing portion 2 and the second needle 13, and to permit the second needle 13 to slide towards the retracted position. The position of the second needle 13 within the lumen of the first needle 1 beneficially means that there is only one puncture wound formed in the patient's skin, as the first needle 12 will enlarge the puncture wound formed by the second needle 13 during use.

In alternative examples the second needle 13 does not pass into, or through, the lumen of the first needle 12. Instead, the second needle 13 can extend through another part of the handle 11, for example adjacent to the first needle 12.

In the above-described examples the second needle 13 is initially in an extended position, and the first and second needles 12, 13 can be simultaneously positioned in the patient. FIG. 8 illustrates an alternative example in which the second needle 13 is initially in a retracted position. In this example, as shown, the actuation tab 4 extends from the handle 11. In the retracted position the second needle 13 may be within the first needle 12, as illustrated, or alongside the first needle 12 within the handle 11. The electrode lead 3 is partially within the second needle 13 and looped between the housing portion 2 and the second needle 13.

During implantation, the first needle 12 is inserted into the patient with the implant delivery device 10 in the configuration shown in FIG. 8, then the actuation tab 16 is pushed towards the handle 11 to extend the second needle 13 beyond the first needle 12 and carry the electrode lead 3 into position. In this position the locking mechanism 17 can be engaged such that the actuation tab 16 and second needle 13 are locked in position.

In some examples, the implant delivery device 10 may be removed from the patient with the second needle 13 in the extended position, and a deployment member (e.g., a pusher) may be provided to urge the medical implant 1 out of the first and second needles 12, 13. In other examples, after being extended the second needle 13 can then be retracted by pulling the actuation tab 16 away from the handle 11 in the manner described above with reference to FIGS. 5A to 5C, to release the electrode lead 3 and housing portion 2.

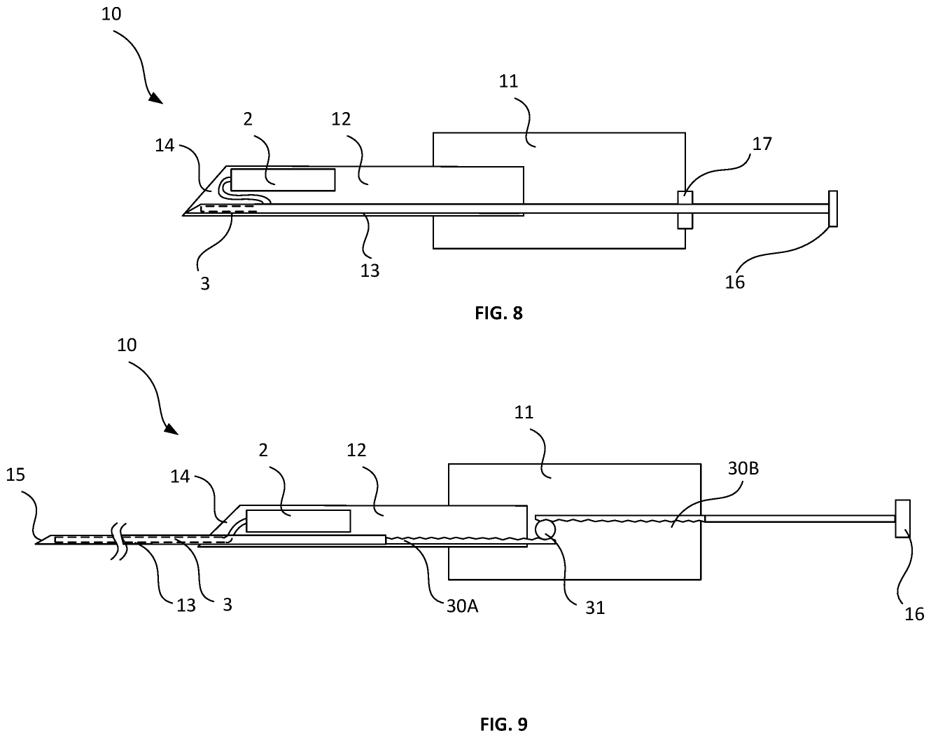

FIG. 9 illustrates an alternative example delivery device 10. In this example the delivery device 10 comprises a handle 11, a first needle 12 holding the housing portion 2 and a second needle 13 holding the electrode lead 3, as previously described. In this example, the actuation tab 16 is arranged to be pushed towards the handle 11 (and the patient) in order to retract the second needle 13.

In particular, as illustrated, the implant delivery device 10 has a rack and pinion mechanism for translating movement of the actuation tab 16 towards the handle 11 into retraction of the second needle 13 (i.e., the second needle 13 moves in an opposite direction to the actuation tab 16).

As shown in FIG. 9, the rack and pinion mechanism comprises a first rack portion 30A attached to, or formed as part of, the second needle 13. The rack and pinion mechanism also comprises a second rack portion 30B attached to, or formed as part of, the actuation tab 16. A pinion gear 31 is rotatably mounted within the handle 11 and is engaged with both the first and second rack portions 30A, 30B. The pinion gear 31 may be mounted within the first needle 12 or, as illustrated, behind the end of the first needle 12 within the handle 11. Accordingly, as the actuation tab 16 is pushed towards the patient the rack and pinion mechanism causes the second needle 13 to be retracted away from the patient to release the electrode lead 3.

Advantageously, pushing the actuation tab 16, rather than pulling the actuation tab 16, may permit one-handed operation of the implant delivery device 10.

It will be appreciated that in some examples the delivery device 10 described above may be adapted to implant the medical implant illustrated in FIG. 3B. In these examples the second needle 13 may be omitted, so that the delivery sheath comprises only the first needle 12, which holds the medical implant 1. In such examples, implantation is achieved by percutaneously positioning the first needle 12 and then ejecting, for example pushing, the medical implant 1 out of the first needle 12.

It will be appreciated that in some examples the delivery device 10 described above may be adapted to implant the medical implant illustrated in FIG. 3C. In these examples the second needle 13 may hold the wire 77 and wireless power transmitter 75, and the first needle 12 may hold the housing portion 2. The second needle 13 can be retractable, as described above, to implant the wire 77 and wireless power transmitter 75.

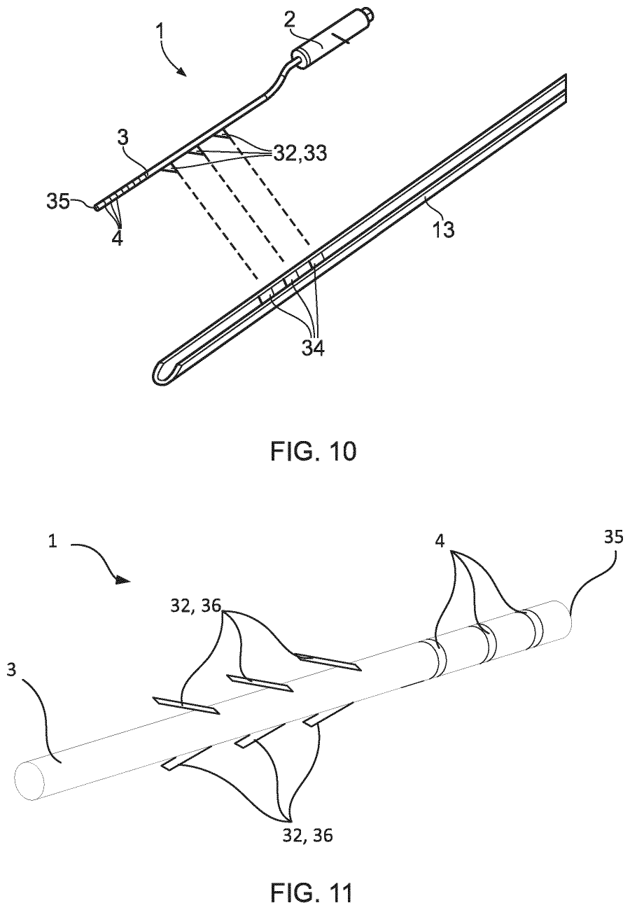

The medical implant 1 includes one or more anti-migration members 32 adapted to hold the medical implant 1 in position once delivered to the location shown in FIG. 2. FIGS. 10 to 23 illustrate different example anti-migration members 32. FIGS. 10 to 23 illustrate different anti-migration members 32 with reference to the medical implant 1 of FIG. 3A, with a housing portion 2 and an elongate electrode lead 3 having electrodes 4. Each of the anti-migration members 32 may be provided on the electrode lead 3 and/or on the housing portion 2. However, it will be appreciated that the anti-migration members 32 described below may be provided on the medical implant 1 described with reference to FIG. 3B, in which case the anti-migration members 32 are provided on the housing portion 2. Further, the anti-migration members 32 described below may be provided on the medical implant 1 described with reference to FIG. 3C, in which case the anti-migration members 32 may be provided on the housing portion 2 and/or the wire 77 and/or the wireless power transmitter 75.

In each of the examples of FIGS. 10 to 22 the anti-migration member 32 is resiliently biased and accommodated within the delivery sheath prior to deployment of the medical implant 1. As the medical implant 1 is released from the delivery device 10 the resiliently biased anti-migration member 32 moves to a deployed position where it acts to hold the medical implant 1 in position within the patient's tissue.

In the example of FIG. 10 the medical implant 1, in particular the electrode lead 3, comprises one or more (in this example three) anti-migration members 32. In this example, the anti-migration members 32 are resiliently biased fins 33 protruding from a side of the electrode lead 3. The fins 33 are angled towards the distal tip 35 of the electrode lead 3. The fins 33 are made from a resiliently deformable material, for example a metal (stainless steel), aluminium, or Nitinol, or a biocompatible polymer. The fins 33 may be thin, sheet-like protrusions. The fins 33 may be integrated into the electrode lead 3, in particular a casing or the electrode lead 3, or may be attached by a collar or clamp.

The angled shape of the fins 33 provides for deformation of the fins 33 towards the electrode lead 3 as the electrode lead 3 moves into the patient's tissue and when the electrode lead 3 is received in the delivery sheath of the delivery device. The fins 33 also act to prevent pull-out of the electrode lead 3 away from the patient.

In examples, the fins 33 may be folded against the side of the electrode lead 3 within the delivery sheath. In this way, as the electrode lead 3 moves out of the delivery sheath (e.g., as the second needle 13 is retracted) the fins 33 unfold (due to their resilience) to the position shown in FIG. 10.

In other examples, the fins 33 may be received in recesses or openings 34 formed in the delivery sheath (e.g., second needle 13, as shown). The recesses 34 may accommodate the fins 33 and the fins 33 deform as they are moved out of the recesses 34 during retraction of the second needle 13. Similarly, openings 34 may accommodate the fins 33 such that the fins 33 protrude through the second needle 13, and the fins 33 can deform as they are moved out of the openings 34 during implantation.

In some examples, the fins 33 may be additionally or alternatively provided on the housing portion 2 of the medical implant. In some examples, the fins 33 may be provided on the housing portion of the medical implant 1 of FIG. 3B. In some examples, the fins 33 may be provided on the housing portion 2, wire 77, or wireless power transmitter 75 of the medical implant 1 of FIG. 3C.

In the example of FIG. 11 the medical implant 1, in particular the electrode lead 3, comprises one or more (in this example three) anti-migration members 32. In this example, the anti-migration members 32 are resiliently biased tines 36 protruding from a side of the electrode lead 3. In this example the tines 36 are arranged in pairs located on opposing sides of the electrode lead 3. The tines 36 are attached to the electrode lead 3 at one end and protrude lengthwise along the electrode lead 3, away from the distal tip 35 of the electrode lead 3. The tines 36 are made from a resiliently deformable material, for example a metal (stainless steel), aluminium, or Nitinol, or a biocompatible polymer. The tines 36 are thin, sheet-like protrusions. The tines 36 are resiliently biased to the position illustrated in FIG. 11 in which they protrude from the electrode lead 3 at an angle.

The tines 36 may be integrated into the electrode lead 3, in particular a casing or the electrode lead 3. In other examples, the tines 36 may extend from a collar that is attached to the electrode lead 3, or they may be clamped to the electrode lead 3.

The orientation of the tines 36 provides for deformation of the tines 36 towards the electrode lead 3 as the electrode lead 3 moves into the patient's tissue. The tines 36 also act to prevent pull-out of the electrode lead 3 away from the patient.

In examples, the tines 36 may be folded against the side of the electrode lead 3 within the delivery sheath (e.g., the second needle 13) prior to deployment. In this way, the tines 36 move outward (due to their resilience) to the position shown in FIG. 11 during implantation.

In examples, the tines 36 may be received in recesses or openings formed in the delivery sheath (e.g., second needle) in the same manner as the fins 33 described with reference to FIG. 10.

In some examples, the tines 36 may be additionally or alternatively provided on the housing portion 2 of the medical implant. In some examples, the tines 36 may be provided on the housing portion of the medical implant 1 of FIG. 3B. In some examples, the tines 36 may be provided on the housing portion 2, wire 77, or wireless power transmitter 75 of the medical implant 1 of FIG. 3C.

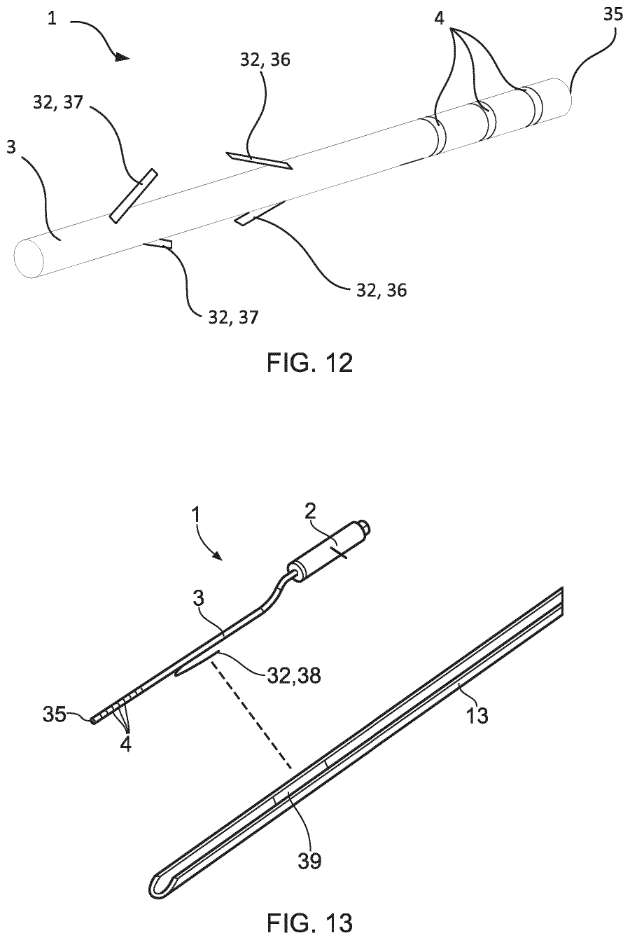

The example of FIG. 12 is similar to that of FIG. 11, with tines 36 provided on the electrode lead 3, housing portion 2, wire 77 or wireless power transmitter 75 of the medical implant 1. In this example, a second set of tines 37 are provided in opposite orientation to the tines 36 to prevent movement of the medical implant 1 in both directions.

In the example of FIG. 13 the medical implant 1, in particular the electrode lead 3, comprises an anti-migration member 32. In this example, the anti-migration member 32 is a resiliently biased hook 38 protruding from a side of the electrode lead 3. The hook 38 is attached to the electrode lead 3 at one end and protrudes initially towards the distal tip 35 and then in a generally opposing direction, forming a hook. The hook 38 is made from a resiliently deformable material, preferably a shape memory material, such as spring stainless steel or Nitinol. The hook 38 is resiliently biased to the position illustrated in FIG. 13 in which it protrudes from the electrode lead 3 at an angle.

The hook 38 may be integrated into the electrode lead 3, in particular a casing or the electrode lead 3. In other examples, the hook 38 may extend from a collar that is attached to the electrode lead 3, or may be clamped to the electrode lead 3.

The orientation of the hook 38 provides for deformation of the hook 38 towards the electrode lead 3 as the electrode lead 3 moves into the patient's tissue. When deformed inwards, the hook 38 lies flat against the side of the electrode lead 3. When deployed as shown in FIG. 13 the hook 38 acts to prevent pull-out of the electrode lead 3 away from the patient.

In examples, the hook 38 may be folded against the side of the electrode lead 3 within the delivery sheath (e.g., second needle 13 as illustrated) prior to deployment. In this way, the hook 38 moves outward (due to its resilience) to the position shown in FIG. 13 during implantation.

In examples, the hook 38 may be received in a recess or opening 39 formed in the delivery sheath, e.g., within the second needle 13.

In some examples, the hook 38 may be additionally or alternatively provided on the housing portion 2 of the medical implant. In some examples, the hook 38 may be provided on the housing portion of the medical implant 1 of FIG. 3B. In some examples, the hook 38 may be provided on the housing portion 2, wire 77, or wireless power transmitter 75 of the medical implant 1 of FIG. 3C.

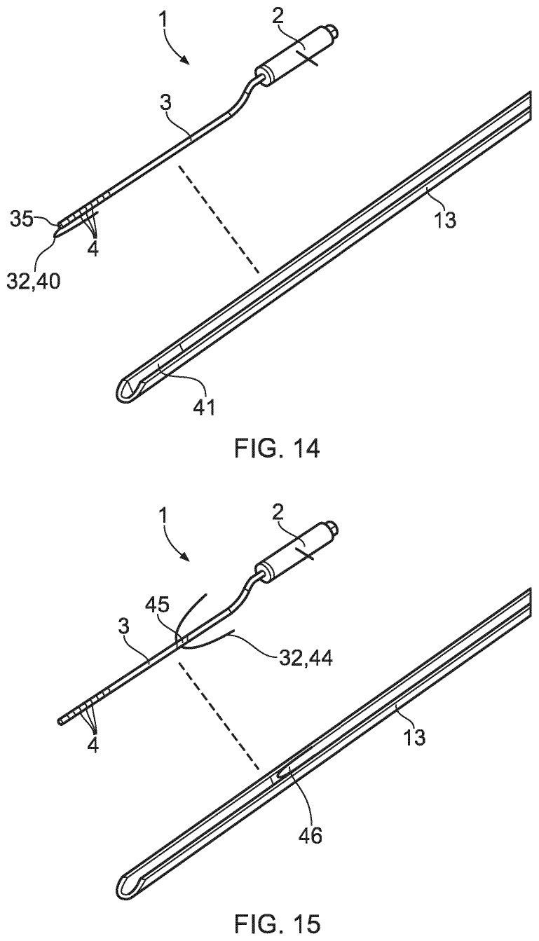

The example of FIG. 14 is similar to that of FIG. 13, but the hook 40 is provided at the distal tip 35 of the electrode lead 3. The hook 40 may fold against the side of the electrode lead 3 within the delivery sheath (e.g., the second needle 13), and/or it may be received in a recess or opening 41 formed in the delivery sheath (e.g., the second needle 13).

The example of FIG. 15 is similar to that of FIG. 13, where the anti-migration member 32 comprises a resiliently biased hook 44 attached to the electrode lead 3. In this example, the anti-migration member 32 comprises two resiliently biased hooks 44 attached to the electrode lead 3 by a collar 45. The anti-migration member 32 is received in a recess or opening 46 formed in the delivery sheath (e.g., the second needle 13) as shown.

In some examples, the resiliently biased hooks 44 may be additionally or alternatively provided on the housing portion 2 of the medical implant 1. In some examples, the resiliently biased hooks 44 may be provided on the housing portion of the medical implant 1 of FIG. 3B. In some examples, the resiliently biased hooks 44 may be provided on the housing portion 2, wire 77, or wireless power transmitter 75 of the medical implant 1 of FIG. 3C.

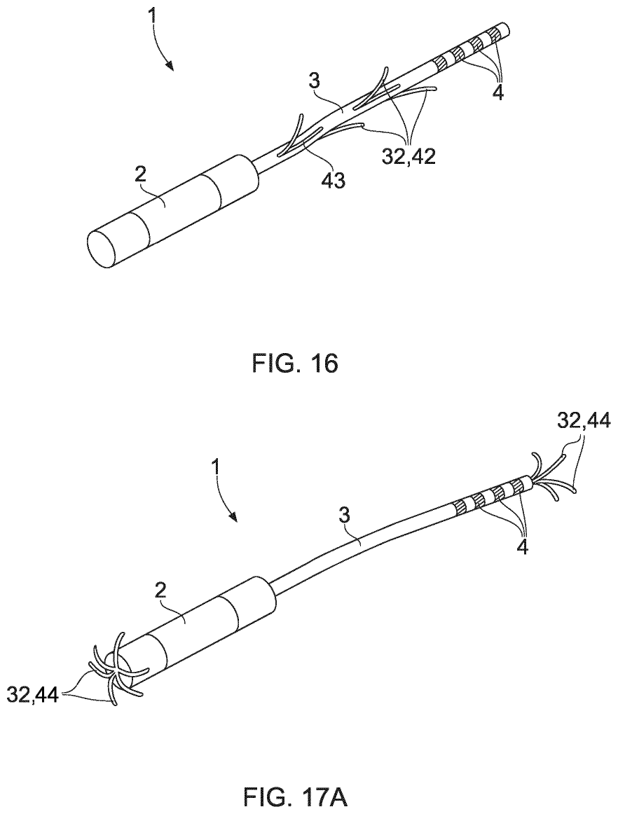

In the example of FIG. 16 the medical implant 1, in particular the electrode lead 3, comprises an anti-migration member 32. In this example, the anti-migration member 32 is a resiliently biased tine 42 formed from an outer layer of the electrode lead 3. In particular, the electrode lead 3 comprises an outer layer and the resiliently biased tine 42 is formed by a U-shaped cut in the outer layer, which permits the tine 42 to resiliently deform outwards. As shown, when the resiliently biased tine 42 is deployed a recess 43 is left in the outer layer of the electrode lead 3. Accordingly, when the electrode lead 3 is received in the delivery sheath (e.g., the second needle 13, see FIG. 4) the resiliently biased tine 42 is held in the recess 43. In the illustrated example there are a plurality of resiliently biased tines 42, in particular four resiliently biased tines 42. The resiliently biased tines 42 are spaced along the length of the electrode lead 3, and may additionally or alternatively be radially spaced about the electrode 3. Accordingly, the resiliently biased tines 42 move outward and act to prevent movement of the electrode lead 3 during implantation.

In examples, the outer layer of the electrode lead 3 (and also the resiliently biased tines 42) may be made from a metal, such as steel, or from a biocompatible polymer.

In some examples, the resiliently biased tines 42 may be additionally or alternatively provided on the housing portion 2 of the medical implant 1. In some examples, the resiliently biased tines 42 may be provided on the housing portion of the medical implant 1 of FIG. 3B. In some examples, the resiliently biased tines 42 may be provided on the housing portion 2, wire 77, or wireless power transmitter 75 of the medical implant 1 of FIG. 3C.

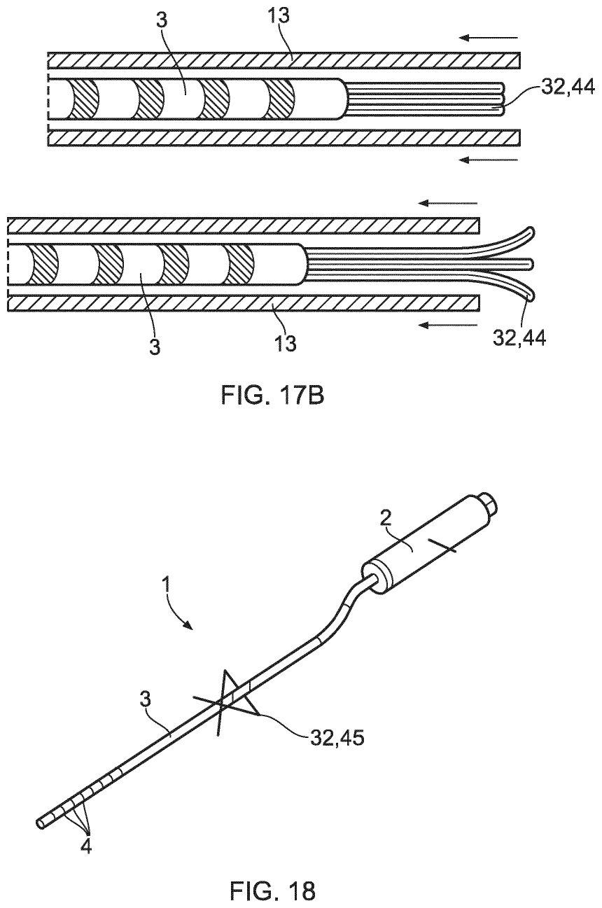

In the example of FIGS. 17A and 17B the medical implant 1 comprises one or more anti-migration members 32. In this example, the anti-migration members 32 comprise a plurality of resiliently biased hooks 44 formed at the end of the electrode lead 3 and/or at the end of the housing portion 2 opposite to the electrode lead 3. FIGS. 17A and 17B illustrate both options. As shown, a plurality of resiliently biased hooks 44 are formed at the end of the medical implant 1 and have different directions to as to be radially spaced relative to the medical implant 1. The plurality of resiliently biased hooks 44 are resilient and biased towards a bent position, as shown in FIG. 17A, in which they act to anchor the medical implant 1 in the patient's tissue.

As shown in FIG. 17B, before deployment the plurality of resiliently biased hooks 44 are housed within the delivery sheath (e.g., the second needle 13) in a deflected state (i.e., straight). When the medical implant 1 is deployed the plurality of resiliently biased hooks 44 return to their biased position as also shown in FIG. 17B.

Advantageously, locating the anti-migration member 32 at the end of the medical implant 1 can avoid any increase in diameter of the medical implant 1 and accompanying delivery device. In addition, providing an anti-migration member 32 on both the electrode lead 3 and the housing portion 2 can improve anchoring as both ends of the medical implant 1 are anchored.

In some examples, the anti-migration members 32 may be provided on the housing portion 2 of the medical implant 1 of FIG. 3B, for example on both ends of the housing portion 2. In some examples, the anti-migration members 32 may be provided on one or more of the housing portion 2, wire 77, or wireless power transmitter 75 of the medical implant 1 of FIG. 3C.

In the example of FIG. 18 medical implant 1, in particular the electrode lead 3, comprises an anti-migration member 32. In this example, the anti-migration member 32 is a resiliently biased frame 45 having a collapsed configuration in which the resiliently biased frame 45 can be housed within the delivery sheath (e.g., the second needle 13, see FIG. 4), and an expanded configuration shown in FIG. 18. The resiliently biased frame 45 is biased towards the expanded configuration. The resiliently biased frame 45 may be made from a polymer or a metal, and preferably a memory material such as Nitinol.

Accordingly, when the medical implant 1 is deployed the resiliently biased frame 45 will expand into the expanded configuration and anchor the medical implant 1. As illustrated, the resiliently biased frame 45 is provided on the electrode lead 3 and may be attached to the electrode lead 3 by a collar, clamp or similar. The resiliently biased frame 45 is positioned between the housing portion 2 and the electrodes 4 so that the delivery sheath (e.g., the second needle 13, see FIG. 4) can be partially retracted to expose the electrodes 4 without the resiliently biased frame 45 being deployed. Accordingly, the position of the electrodes 4 can be tested before the anti-migration member 32 is deployed. In alternative examples the anti-migration member 32 is provided on the housing portion 2 in addition to on the electrode lead 3 or as well as on the electrode lead 3.

In some examples, the anti-migration members 32 may be provided on the housing portion 2 of the medical implant 1 of FIG. 3B. In some examples, the anti-migration members 32 may be provided on one or more of the housing portion 2, wire 77, or wireless power transmitter 75 of the medical implant 1 of FIG. 3C.

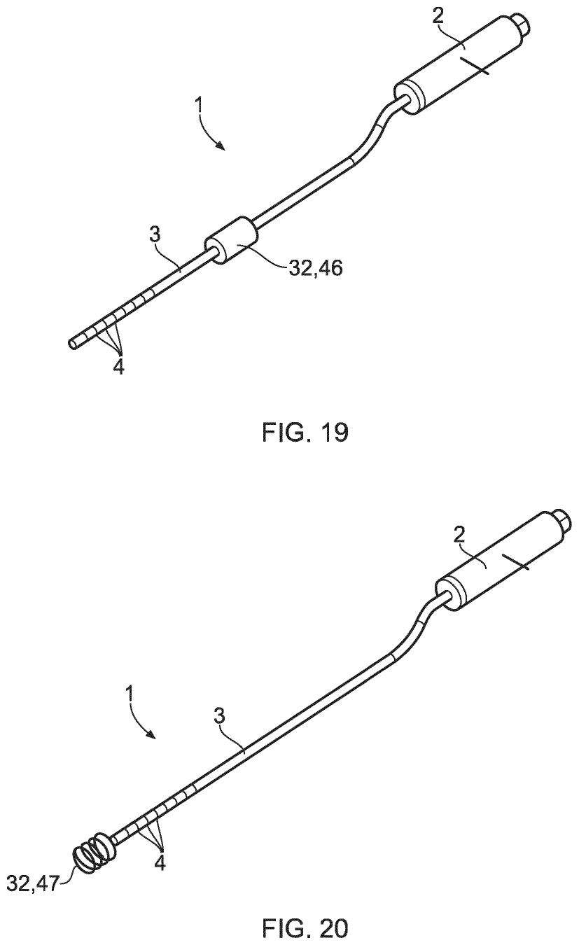

In the example of FIG. 19 medical implant 1, in particular the electrode lead 3, comprises an anti-migration member 32. In this example, the anti-migration member 32 is a resiliently biased stent 46 having a collapsed configuration in which the resiliently biased stent 46 can be housed within the delivery sheath (e.g., the second needle 13, see FIG. 4), and an expanded configuration shown in FIG. 19. The resiliently biased stent 46 is biased towards the expanded configuration. The resiliently biased stent 46 may comprise one or more struts and/or rings in the conventional manner of, for example, a vascular stent. The resiliently biased stent 46 may be made from a biocompatible polymer or a metal, and preferably a memory material such as Nitinol.

Accordingly, when the medical implant 1 is deployed the resiliently biased stent 46 will expand into the expanded configuration and anchor the medical implant 1. As illustrated, the resiliently biased stent 46 is provided on the electrode lead 3 and may be attached to the electrode lead 3 by a collar or similar. The resiliently biased stent 46 is positioned between the housing portion 2 and the electrodes 4 so that the delivery sheath (e.g., the second needle 13, see FIG. 4) can be partially retracted to expose the electrodes 4 without the resiliently biased stent 46 being deployed. Accordingly, the position of the electrodes 4 can be tested before the anti-migration member 32 is deployed. In alternative examples the anti-migration member 32 is provided on the housing portion 2 in addition to on the electrode lead 3 or as well as on the electrode lead 3.

In some examples, the anti-migration members 32 may be provided on the housing portion 2 of the medical implant 1 of FIG. 3B. In some examples, the anti-migration members 32 may be provided on one or more of the housing portion 2, wire 77, or wireless power transmitter 75 of the medical implant 1 of FIG. 3C.

In the example of FIG. 20 medical implant 1, in particular the electrode lead 3, comprises an anti-migration member 32. In this example, the anti-migration member 32 is a resiliently biased coil 47 having a collapsed configuration in which the resiliently biased coil 47 can be housed within the delivery sheath (e.g., the second needle 13, see FIG. 4), and an expanded configuration shown in FIG. 20. The resiliently biased coil 47 is biased towards the expanded configuration. The resiliently biased coil 47 may be made from a biocompatible polymer or a metal, and preferably a memory material such as Nitinol.

Accordingly, when the medical implant 1 is deployed the resiliently biased coil 47 will expand into the expanded configuration and anchor the medical implant 1. As illustrated, the resiliently biased coil 47 is provided on the electrode lead 3 and may be attached to the electrode lead 3 by a collar, clamp or similar.

In the illustrated example the resiliently biased coil 47 is located at the end of the electrode lead 3. In other examples the resiliently biased coil 47 may be positioned between the housing portion 2 and the electrodes 4 so that the delivery sheath (e.g., the second needle 13, see FIG. 4) can be partially retracted to expose the electrodes 4 without the resiliently biased coil 47 being deployed. Accordingly, the position of the electrodes 4 can be tested before the anti-migration member 32 is deployed. In alternative examples the anti-migration member 32 is provided on the housing portion 2 in addition to on the electrode lead 3 or as well as on the electrode lead 3.

In some examples, the anti-migration members 32 may be provided on the housing portion 2 of the medical implant 1 of FIG. 3B, for example on both ends of the housing portion 2. In some examples, the anti-migration members 32 may be provided on one or more of the housing portion 2, wire 77, or wireless power transmitter 75 of the medical implant 1 of FIG. 3C.

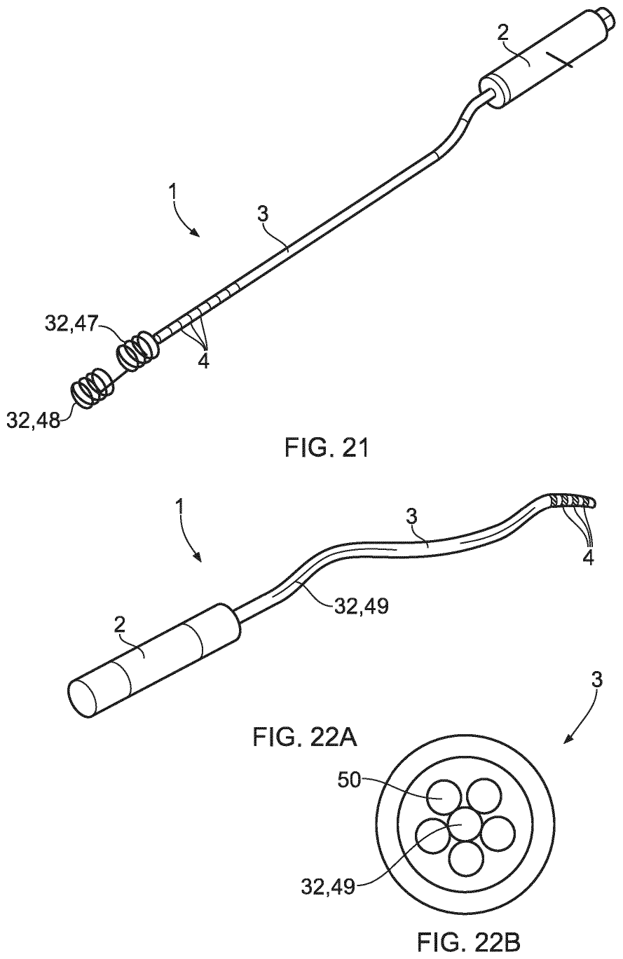

The example of FIG. 21 is similar to the example of FIG. 20, where the anti-migration member 32 comprises a resiliently biased coil 47. However, in the example of FIG. 21 the anti-migration member 32 further comprises second resiliently biased coil 48 attached to the resiliently biased coil 47. The second resiliently biased coil 48 is formed in the same way as the resiliently biased coil 47 described above, and is connected by a straight portion. The second resiliently biased coil 48 improves anchoring of the medical implant 1.

In the example of FIG. 22 the medical implant 1, in particular the electrode lead 3, comprises an anti-migration member 32. In this example, the anti-migration member 32 is a shape memory member 49 extending through the electrode lead 3. In particular, as shown in FIG. 22B, the shape memory member 49 is embedded within the electrode lead 3. The shape memory member 49 may be provided alongside, or between, conductors (wires) 50 in the electrode lead 3. The shape memory member 49 may extend the full length of the electrode lead 3, or partially along the electrode lead 3, or comprise a plurality of parts spaced along the electrode lead 3. The shape memory member 49 may be a polymer or a metal, in particular a shape memory material such as Nitinol. The shape memory member 49 has a non-linear form, for example wavy as illustrated. Accordingly, when unconstrained the electrode lead 3 will adopt a non-linear form that acts to anchor the electrode lead 3 in the patient's tissue. When the electrode lead 3 is constrained in the delivery sheath (e.g., the second needle 13, see FIG. 4) the shape memory member 49 is deformed to be straight. Accordingly, when the medical implant 1 is deployed the shape memory member 49 will deform the electrode lead 3 into a non-linear form and anchor the medical implant 1. The shape memory member 47 also allows the delivery sheath (e.g. the second needle 13, see FIG. 4) to be partially retracted to expose the electrodes 4. The delivery sheath (e.g. the second needle 13, see FIG. 4) can also be re-extended for repositioning the electrode lead 3. Accordingly, the position of the electrodes 4 can be tested before the electrode lead 3 is fully deployed.

In some examples, the shape memory member 47 may be provided in the wire 77 of the medical implant 1 of FIG. 3C.

As described above, the medical implant 1 comprises one or more anti-migration members 32 that act to reduce movement of the medical implant 1 after implantation. Referring to FIGS. 4 to 9, in some examples the medical implant 1 is received in the delivery device 10 and the electrode lead 3 comprises one or more anti-migration members 32. In such examples, the anti-migration members 32 may be non-aligned with the slot 18 formed in the second needle 13. In particular, the second needle 13 may have a retaining portion that acts to hold the electrode lead 3 and retain the anti-migration members 32.

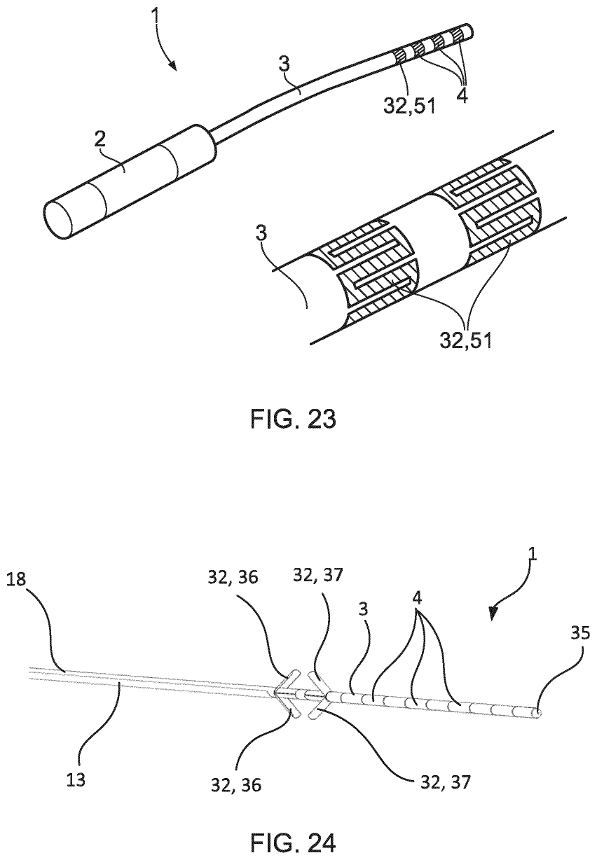

In the example of FIG. 23 the medical implant 1, in particular the electrode lead 3, comprises an anti-migration member 32. In this example, the anti-migration member 32 is a high friction area 51 formed on the surface of the electrode lead 3. The high friction area 51 may comprise a coating or collar. The high friction area 51 may have a relatively high surface roughness, for example formed by grooves. The high friction area 51 can thereby help to prevent movement of the electrode lead 3 relative to the patient's tissue once the medical implant 1 has been implanted. The electrode lead 3 may comprise more than one high friction area 51 spaced along the electrode lead 3, for example two as illustrated.

In the illustrated example the high friction area 51 is located between the housing portion 2 and the electrodes 4 so that the delivery sheath (e.g., the second needle 13, see FIG. 4) can be partially retracted to expose the electrodes 4 without the high friction area 51 being exposed. Accordingly, the position of the electrodes 4 can be tested before fully deploying the electrode lead 3. In alternative examples the anti-migration member 32 is provided on the housing portion 2 in addition to on the electrode lead 3 or as well as on the electrode lead 3.

In some examples, the high friction area 51 may be provided on the housing portion 2 of the medical implant 1 of FIG. 3B. In some examples, the high friction area 51 may be provided on one or more of the housing portion 2, wire 77, or wireless power transmitter 75 of the medical implant 1 of FIG. 3C.

In the example of FIG. 24 the medical implant 1, in particular the electrode lead 3, comprises an anti-migration member 32. In this example, the anti-migration member 32 is similar to that of FIG. 12, with a first set of tines 36 and a second set of tines 37 arranged in opposite orientations on the electrode lead 4. The tines 36, 37 are resiliently biased. In this example the anti-migration member 32 is disposed such that the electrodes 4 are arranged between the anti-migration member 32 and the tip 35 as described above. In this example, the tines 37 have a width that is greater than the opening (slot) 18 in the second needle 13. Accordingly, the tines 36, 37 are held against the electrode lead 3 within the second needle 13 and cannot be deployed even if they are aligned with the opening 19. When the second needle 13 is retracted, as described above, the tines 36, 37 spring outwards to anchor the electrode lead 3 in the patient's tissue. The electrodes 4 are disposed between the tip 35 of the electrode lead 3 and the anti-migration member 32 so that, as with above examples, the second needle 13 can be partially retracted to expose the electrodes 4, for example to test the implant position of the electrodes 4, without deploying the anti-migration member 32, Accordingly, if needed, the second needle 13 can be redeployed and the electrode lead 4 can be repositioned.

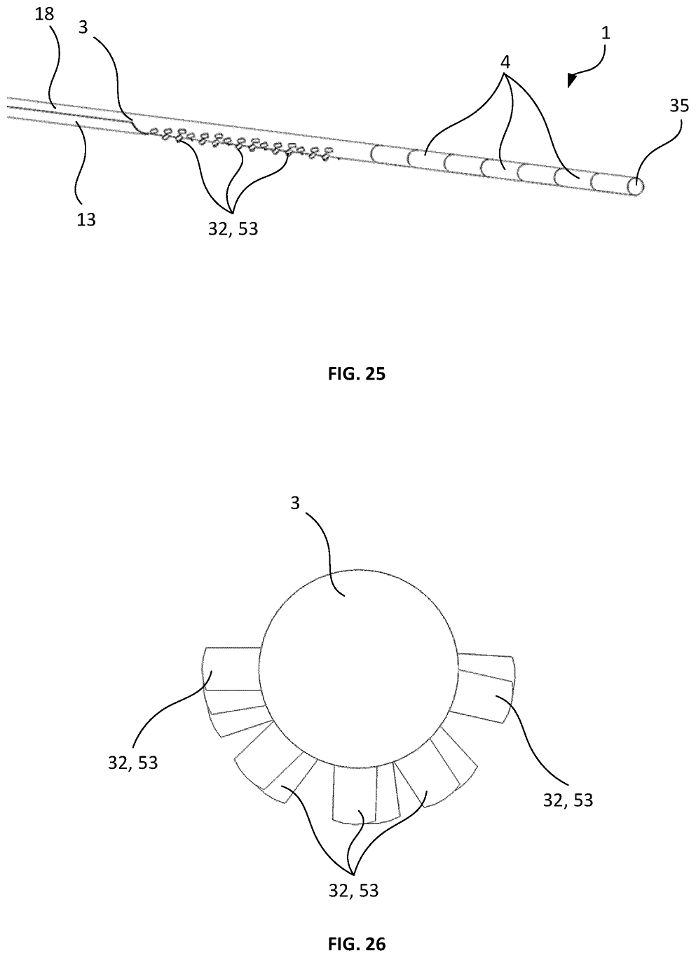

FIG. 26 shows an end view of the example medical implant 1 of FIG. 25. In this example the medical implant 1, in particular the electrode lead 3, comprises an anti-migration member 32. In this example, the anti-migration member 32 includes a plurality of resiliently biased tines 53 arranged on one side of the electrode lead 3. The resiliently biased tines 53 are disposed so as not to align with the opening 18 in the second needle 13 when the electrode lead 3 is held in the second needle 13. In particular, as shown in FIG. 26, the resiliently biased tines 53 are all arranged to extend from the electrode lead 3 within about 180 degrees of each other. When the electrode lead 3 is held in the second needle 13 none of the tines 53 are aligned with the opening 18, so all are held against the electrode lead 3 in a retracted state. In some examples, the tines 53 may be attached to the electrode lead 3 about an angle more or less than about 180 degrees, and in particular the tines 53 may be attached to the electrode lead 3 about an angle that is less than the size of the opening 18. When the second needle 13 is retracted, as described above, the tines 53 spring outwards to anchor the electrode lead 3 in the patient's tissue. As with above examples, the second needle 13 can be partially retracted to expose the electrodes 4, for example to test the implant position of the electrodes 4, without deploying the anti-migration member 32, Accordingly, if needed, the second needle 13 can be redeployed and the electrode lead 4 can be repositioned.

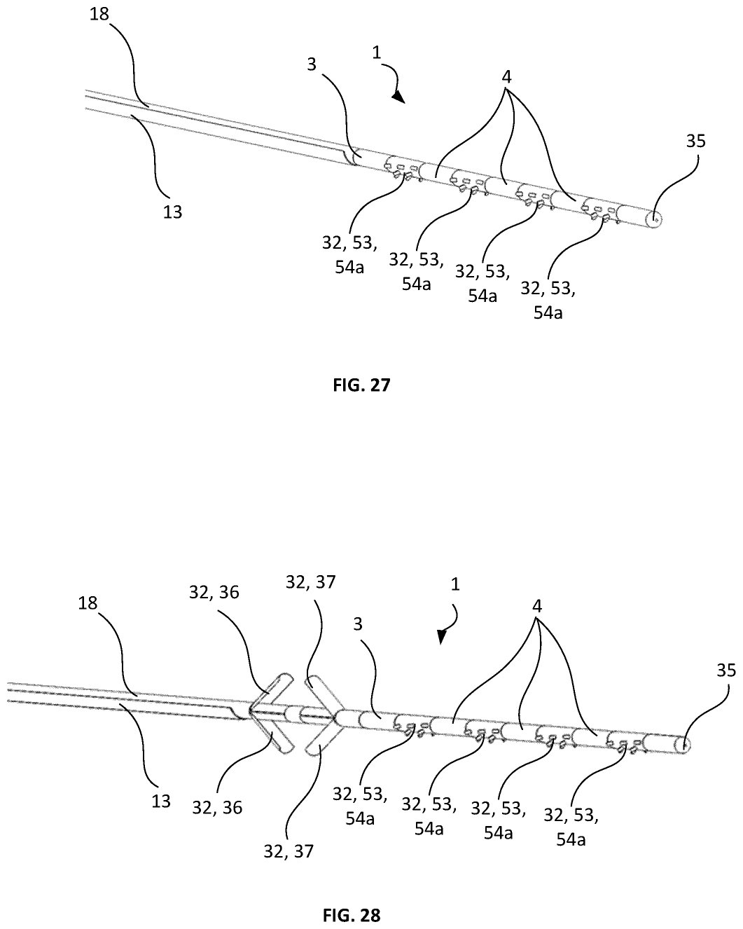

In the example of FIG. 27 the anti-migration member 32 is similar that of FIGS. 25 and 26, with resiliently biased tines 53 disposed so as not to align with the opening 18 in the second needle 13 when the electrode lead 3 is held in the second needle 13. In this example the tines 53 are arranged in groups 54a-54d that are disposed between adjacent electrodes 4.

The example of FIG. 28 is a combination of FIGS. 24 and 27. In particular, the electrode lead 3 of the medical implant 1 includes an anti-migration member 32 formed of the first set of tines 36 and the second set of tines 37 arranged in opposite orientations on the electrode lead 4, as well as the groups 54a-54d of tines 53 interposed between the electrodes 4.

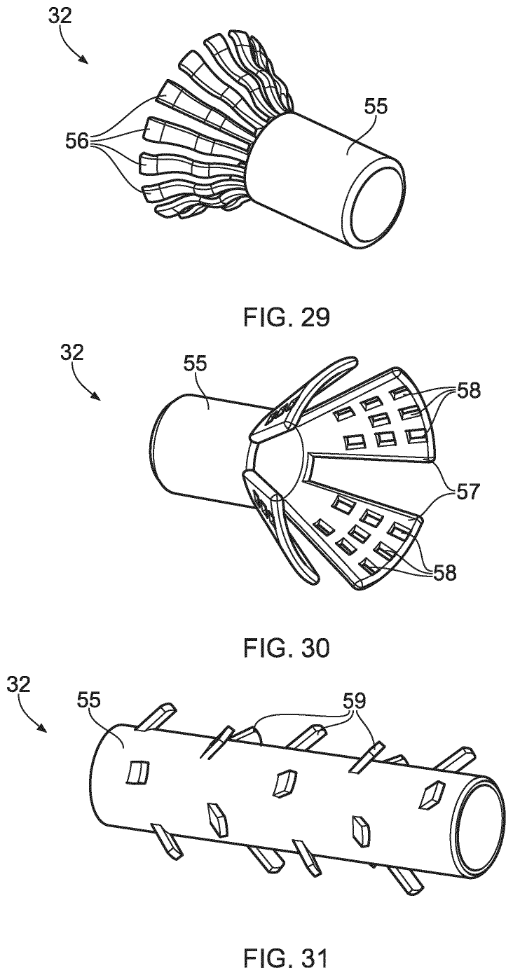

FIGS. 29 to 31 show examples of anti-migration members 32 that can be attached to the electrode lead 3 of the medical implant 1, for example by sliding over the electrode lead 3 and attaching to the electrode lead 3 by friction fit, adhesive or welding. In other examples the anti-migration members 32 can be over-moulded onto the electrode lead 3.

In the example of FIG. 29 the anti-migration member 32 includes a sleeve portion 55 that slides over, or is over-moulded on, the electrode lead. A plurality of resiliently biased tines 56 extend from the sleeve portion 55, in particular from an end of the sleeve portion 55. The tines 56 extend from an end of the sleeve portion 55. The resiliently biased tines 56 have a wavey (non-linear) shape to improve anchoring.

The example of FIG. 30 is similar to that of FIG. 29, except that the resiliently biased tines 57 have openings 58. The openings 58 may help with long term anchorage of the electrode lead 3 by allowing tissue ingrowth through the openings 58. In some examples, each resiliently biased tine 57 may have a single opening 58 or a plurality of openings 58. The openings 58 may be round, square, rectangular, or other shape.

In addition, in the example of FIG. 30 the resiliently biased tines 57 are narrower where they are attached to the sleeve portion 55 and wider at the free ends. The maximum width of the resiliently biased tines 57 (at the free ends) is greater than the width of the opening to prevent deployment of the resiliently biased tines 57 before retraction of the second needle. The edge of the tines 57 between the two ends may be straight, as illustrated, or may be non-linear, for example curved or wavy. The width of the tines 57 may therefore change in at non-constant rate between the two ends of the tines 57. The resiliently biased tines 57 are spaced from each other at the sleeve portion 55 so that there is space between the resiliently biased tines 57 for them to fold inwards when received in the second needle. In this example the resiliently biased tines 57 are curved. The curvature may match the outer diameter of the electrode lead so that when the tines 57 are held against the electrode lead 3 they form a close fit with the electrode lead 3.

The example of FIG. 31 is similar to that of FIG. 29, except that the resiliently biased tines 59 extend from a side surface of the sleeve portion 55 rather than an end of the sleeve portion 55.

In any of the above-described examples the anti-migration member 32 may include openings like those shown in FIG. 30, or may be porous, to allow tissue ingrowth that will improve anchorage of the electrode lead 3.

It will be appreciated that in various examples any of the anti-migration members 32 described above may be attached to the electrode lead 3 using a sleeve portion 55 like that shown in FIGS. 29 to 31. The sleeve portion 55 may be slid over the electrode lead 3. The sleeve portion 55 may be secured to the electrode lead 3 by adhesive, welding, crimping, or by a friction fit. In some examples the sleeve portion 55 (and anti-migration member(s) 32) may be overmoulded on the electrode lead 3.

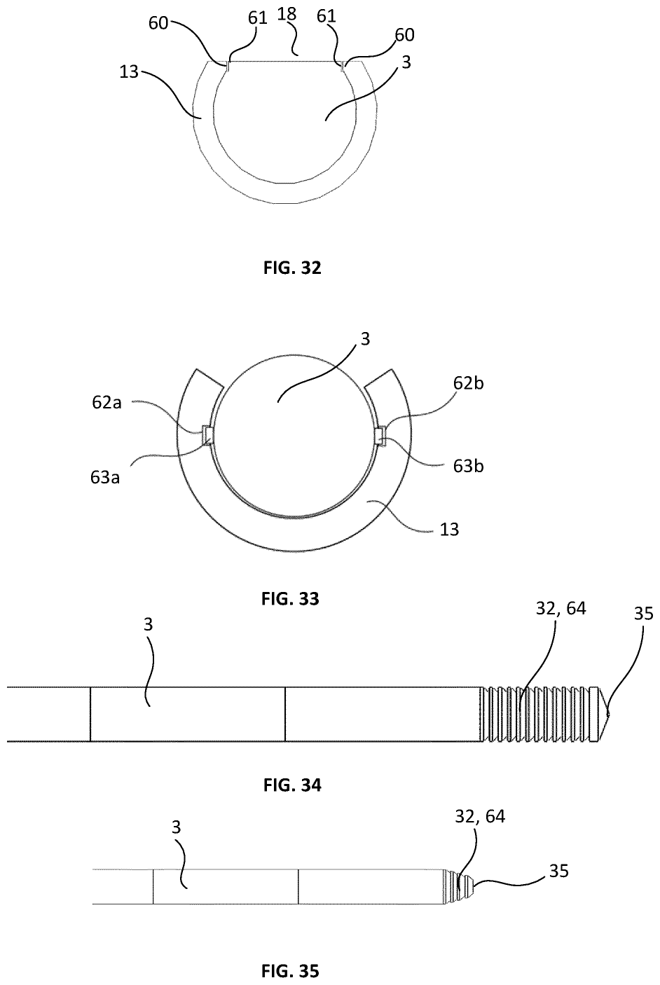

In the examples of FIGS. 10 to 29 described above it may be advantageous to prevent rotation of the electrode lead 3 within the second needle 13, for example to prevent alignment of the anti-migration member 32 with the opening 18 in the second needle 13 before retraction of the second needle 13. FIGS. 32 and 33 illustrate examples of second needles 13 and electrode leads 3 with anti-rotation features to achieve this.

In the example of FIG. 32 the electrode lead 3 has two flat portions 61 that are arranged to abut two flat surfaces 60 of the second needle 13. In this example the flat surfaces 60 are arranged on either side of the opening 18 in the second needle 13. The abutment of the flat surfaces 60 and flat portions 61 prevents rotation of the electrode lead 3 within the second needle 13 and can therefore prevent alignment of the anti-migration member with the opening 18. The flat portions 61 and flat surfaces 60 may extend fully along the electrode lead 3 and second needle 13, or along only a portion of the electrode lead 3 and second needle 13. In examples, the flat portions 61 extend only partially along the electrode lead 3 in the area that corresponds to the anti-migration members.

In the example of FIG. 33, the second needle 13 includes protrusions 63a, 63b extending from a side of the electrode lead 3. The protrusions 63a, 63b may be elongate, extending partially or fully along the length of the electrode lead 3, or they may not be elongate, and may for example be in the form of bosses. Preferably, the protrusions 63a, 63b are aligned with (at least) the anti-migration member on the electrode lead 3. The protrusions 63a, 63b are received in slots 62a, 62b formed on the inner surface of the second needle 13. The slots 62a, 62b and protrusions 63a, 63b co-operate to prevent rotation of the electrode lead 3 within the second needle 13.

FIGS. 34 and 35 illustrate further example electrode leads 3 that include an anti-migration member 32. In these examples the anti-migration member 32 is formed at the tip 35 of the electrode lead 3. The anti-migration member 32 comprises a ridged portion 64 having a plurality of circumferential ridges. The circumferential ridges may be distinct, or may form a spiral. The circumferential ridges of the ridged portion 64 may be angled or tapered, as illustrated, having a tapered surface facing towards the tip 35 and a flat surface facing the other direction. This may improve the anchoring effect of the anti-migration member 32.

Once the electrode lead 3 is implanted the ridged portion 64 may act to prevent the electrode lead 3 from being pulled out of the patient's tissue. After implantation tissue may grow/heal between the ridges to further anchor the electrode lead 3 in the patient's tissue. In the example of FIG. 34 the ridged portion 64 is generally cylindrical, and in the example of FIG. 35 the ridged portion 64 is tapered towards the tip 35.

In the illustrated examples the ridged portions 64 are arranged at the tip 35 of the electrode lead 3. In other examples, one or more ridged portions 64 may be provided, and they may be additionally or alternatively located between the electrodes 4 on the electrode lead 3.

It will be appreciated that the ridged portion(s) 64 may be combined with any of the other example anti-migration members described above.

Throughout the description and claims of this specification, the words “comprise” and “contain” and variations of them mean “including but not limited to”, and they are not intended to (and do not) exclude other components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A neural implant system for percutaneous delivery of a neural implant in a patient's tissue, the neural implant system comprising:
a neural implant having a housing portion and an elongate electrode lead, and
a delivery device comprising:
a handle,
a first needle fixed to the handle and having a lumen adapted to receive the housing portion of the neural implant,
a second needle having a higher gauge than the first needle, the second needle comprising a retaining portion partially surrounding the elongate electrode lead and an open side extending at least partially along the length of the second needle, wherein the second needle is retractably mounted to the housing and retractable to deploy the electrode lead in the patient's tissue,
wherein the electrode lead comprises a resiliently biased anti-migration member aligned with the retaining portion of the second needle so as to be constrained before deployment, and wherein the resiliently biased anti-migration member is configured to move into a deployed position after retraction of the second needle.

2. The neural implant system of claim 1, wherein the retaining portion of the second needle comprises a wall having a recess arranged to receive the resiliently biased anti-migration member.

3. The neural implant system of claim 2, wherein the recess is an opening extending through the wall of the retaining portion.

4. The neural implant system of claim 1, wherein the electrode lead comprises a plurality of resiliently biased anti-migration members.

5. The neural implant system of claim 1, wherein the delivery device comprises a pusher operable to push the housing portion out of the first needle.

6. The neural implant system of claim 1, wherein the neural implant comprises one or more resiliently biased anti-migration members disposed on the housing portion.

7. The neural implant system of claim 6, wherein the first needle comprises a wall having a recess arranged to receive the resiliently biased anti-migration member.

8. The neural implant system of claim 1, wherein the electrode lead comprises an electrode, and wherein the resiliently biased anti-migration member is disposed on the electrode lead between the electrode and the housing portion.

9. The neural implant system of claim 1, wherein the or each resiliently biased anti-migration member comprises a fin.

10. The neural implant system of claim 9, wherein the fin is foldable against the neural implant.

11. The neural implant system of claim 8, wherein the fin is shaped to be angled towards the skin of the user when implanted.

12. The neural implant system of claim 1, wherein the resiliently biased anti-migration member comprises a tine.

13. The neural implant system of claim 12, wherein the resiliently biased anti-migration member comprises a plurality of tines.

14. The neural implant system of 13, wherein a first tine of the plurality of tines is directed in an opposite direction to a second tine of the plurality of tines.

15. The neural implant system of claim 1, wherein the resiliently biased anti-migration member comprises a hook.

16. The neural implant system of claim 15, wherein the hook comprises a shape-memory material, for example a shape-memory metal alloy, such as Nitinol, or a shape-memory polymer.

17. The neural implant system of claim 15, wherein the hook is directed towards the housing portion.

18. The neural implant system of claim 1, wherein the resiliently biased anti-migration member comprises a stent.

19. The neural implant system of claim 18, wherein the stent comprises a collapsible frame that is resiliently biased to an expanded position.

20. The neural implant system of claim 1, wherein the resiliently biased anti-migration member comprises a coil.

21. The neural implant system of claim 1, wherein the resiliently biased anti-migration member is disposed at or near a distal tip of the elongate electrode lead.

22. The neural implant system of claim 1, wherein the resiliently biased anti-migration member comprises a shape-memory member embedded within the electrode lead.

23. The neural implant system of claim 22, wherein the shape-memory member is resiliently biased to a non-linear form and wherein the second needle retains the elongate electrode lead in a substantially linear form before retraction of the second needle.

24. The neural implant system of claim 1, wherein the second needle comprises an opening extending longitudinally along one side of the second needle, wherein the resiliently biased anti-migration member comprises a resiliently biased tine disposed on the electrode lead, and wherein a width of the resiliently biased tine is greater than a width of the opening in the second needle.

25. The neural implant system of claim 24, wherein the resiliently biased tine has a first end attached to the electrode lead and a second end that is a free end, and wherein a width of the resiliently biased tine at the second end is greater than a width of the resiliently biased tine at the first end.

26. The neural implant system of claim 24, wherein the resiliently biased tine is curved.

27. The neural implant system of claim 1, wherein the second needle comprises an opening extending longitudinally along one side of the second needle, wherein the resiliently biased anti-migration member comprises a resiliently biased tine disposed on the electrode lead, and wherein the resiliently biased tine is arranged so as not to align with the opening of the second needle.

28. The neural implant system of claim 1 claim, wherein the resiliently biased anti-migration member comprises a sleeve portion surrounding a part of the electrode lead to secure the resiliently biased anti-migration member to the electrode lead.

29. The neural implant system of claim 1 claim, wherein the second needle and the electrode lead comprise anti-rotation features.

30. The neural implant system of claim 1 claim, wherein the electrode lead further comprises one or more ridged portions.

31. A method of percutaneously delivering a neural implant into tissue of a patient, the method comprising:
providing the neural implant system of claim 1,
percutaneously positioning the second needle in the patient's tissue to a desired anatomical position,
retracting the second needle to release the electrode lead into the tissue, and
deploying the anti-migration member.

32. A medical implant for percutaneous implantation into a patient's tissue by a delivery device, the medical implant comprising a resiliently biased anti-migration member adapted to move from a retracted position when the medical implant is received in the delivery device, to a deployed position when the medical implant is released from the delivery device.

33. The medical implant of claim 32, further comprising a housing portion and an elongate electrode lead extending from the housing portion.

34. The medical implant of claim 33, wherein the resiliently biased anti-migration member is disposed on the electrode lead and/or on the housing portion.

35. The medical implant of claim 32, wherein the resiliently biased anti-migration member comprises one or more of:
a fin protruding from the medical implant,
a tine protruding from the medical implant,
a hook protruding from the medical implant,
a stent protruding from the medical implant,
a coil protruding from the medical implant, and/or
a shape-memory member embedded within the electrode lead.

36. A delivery device for percutaneously implanting a neural implant in a patient's tissue, wherein the neural implant has an housing portion and an elongate electrode lead extending from the housing portion, and wherein the delivery device comprises:
a handle,
a first needle fixed to the handle and having a lumen adapted to receive the housing portion of the neural implant,
a second needle having a higher gauge than the first needle, the second needle comprising a retaining portion partially surrounding the elongate electrode lead and an open side extending at least partially along the length of the second needle, wherein the second needle is retractably mounted to the housing and retractable to deploy the electrode lead in the patient's tissue, and
wherein the retaining portion comprises a recess to accommodate a resiliently biased anti-migration member of the neural implant within the second needle.
